(19) United States
(12) Patent Application Publication
(10) Pub. No.: US 2016/0022995 A1
(43) Pub. Date: Jan. 28, 2016

(54) SYSTEMS, DEVICES, AND METHODS FOR PROVIDING ELECTRICAL STIMULATION THERAPY FEEDBACK

(71) Applicant: Boston Scientific Neuromodulation Corporation, Valencia, CA (US)

(72) Inventors: Sridhar Kothandaraman, Valencia, CA (US); Rafael Carbunaru, Valley Village, CA (US); Dennis Zottola, Ventura, CA (US); Bradley Lawrence Hershey, Valencia, CA (US)

(21) Appl. No.: 14/807,466

(22) Filed: Jul. 23, 2015

Related U.S. Application Data
(60) Provisional application No. 62/028,704, filed on Jul. 24, 2014.

(51) Int. Cl. A61N 1/36

(52) U.S. Cl. CPC A61N 1/36132; A61N 1/36071; A61N 1/36125

(57) ABSTRACT

An electrical stimulation system includes an implantable control module for implantation in a patient's body and having an antenna and a processor coupled to the antenna. The control module provides electrical stimulation signals to an electrical stimulation lead coupled to the implantable control module for stimulation of patient tissue. The system also includes an external programming unit to communicate with the processor of the implantable control module using the antenna and to provide or update stimulation parameters for production of the electrical stimulation signals. The system may also include a patient interface unit to communicate with the external programming unit, the control module, or both. The patient interface unit is configured and arranged to permit a patient to alter one or more of the stimulation parameters within pre-set limits. Alternatively or additionally, the system may also include a database to receive stimulation data from the external programming unit.

### CROSS-REFERENCE TO RELATED APPLICATIONS

This application claims the benefit under 35 U.S.C. §119(e) of U.S. Provisional Patent Application Ser. No. 62/028,704 filed Jul. 24, 2014, which is incorporated herein by reference.

### FIELD

The present invention is directed to the area of implantable electrical stimulation systems and methods of making and using the systems. The present invention is also directed to implantable electrical stimulation systems include devices or methods for providing feedback regarding electrical stimulation, as well as methods of making and using the electrical stimulation systems.

### BACKGROUND

Implantable electrical stimulation systems have proven therapeutic in a variety of diseases and disorders. For example, spinal cord stimulation systems have been used as a therapeutic modality for the treatment of chronic pain syndromes. Peripheral nerve stimulation has been used to treat chronic pain syndrome and incontinence, with a number of other applications under investigation. Functional electrical stimulation systems have been applied to restore some functionality to paralyzed extremities in spinal cord injury patients.

Stimulators have been developed to provide therapy for a variety of treatments. A stimulator can include an implantable pulse generator (IPG), one or more leads, and an array of stimulator electrodes on each lead. The stimulator electrodes are in contact with or near the nerves, muscles, or other tissue to be stimulated. The pulse generator generates electrical pulses that are delivered by the electrodes to body tissue.

### BRIEF SUMMARY

One embodiment is an electrical stimulation system that includes an implantable control module configured and arranged for implantation in a body of a patient and having an antenna and a processor coupled to the antenna. The control module is configured and arranged to provide electrical stimulation signals to an electrical stimulation lead coupled to the implantable control module for stimulation of patient tissue. The system also includes an external programming unit configured and arranged to communicate with the processor of the implantable control module using the antenna and to provide or update stimulation parameters for production of the electrical stimulation signals; and a patient interface unit configured and arranged to communicate with the external programming unit, the control module, or both. The patient interface unit is configured and arranged to permit a patient to alter one or more of the stimulation parameters within pre-set limits.

In at least some embodiments, the system also includes a database configured and arranged to receive stimulation data from the external programming unit, where the stimulation data includes at least one of the stimulation parameters.

In at least some embodiments, the external programming unit, includes a user interface configured and arranged to receive input from a user, and a processor in communication with the user interface and configured and arranged to perform the following actions: a) requesting feedback from the patient, b) receiving patient feedback input at the user interface by the user, and c) in response to the patient feedback, adjusting the stimulation parameters. This external programming unit can be used in any of the embodiments described herein.

In at least some embodiments, the patient interface unit, includes a user interface configured and arranged to receive input from a user, and a processor in communication with the user interface and configured and arranged to perform the following actions: a) requesting feedback from the patient, b) receiving patient feedback input at the user interface by the patient, and c) in response to the patient feedback, adjusting the stimulation parameters. In at least some embodiments, receiving patient feedback includes receiving verbal or visual feedback from the patient, where the user interface is configured and arranged to receive and record the verbal or visual feedback. In at least some embodiments, receiving patient feedback includes presenting an anatomical depiction on the user interface for the patient to provide feedback using the anatomical depiction. Any of these patient interface units can be used in any of the embodiments described herein.

In at least some embodiments, the external programming unit includes a user interface configured and arranged to receive input from a user, and a processor in communication with the user interface and configured and arranged to perform the following actions: a) directing stimulation of the patient over a range of the stimulation parameters, b) receiving patient feedback over the range of the stimulation parameters, and c) presenting an indication of stimulation effects over the range of the stimulation parameters. This external programming unit can be used in any of the embodiments described herein.

In at least some embodiments, the electrical stimulation system is configured and arranged to determine a figure of merit based on feedback from either the patient or a clinician to electrical stimulation provided by the control module. In at least some embodiments, the electrical stimulation system is configured and arranged to determine a figure of merit based on feedback from both the patient and a clinician to electrical stimulation provided by the control module.

Another embodiment is an electrical stimulation system that includes an implantable control module configured and arranged for implantation in a body of a patient and having an antenna and a processor coupled to the antenna. The control module is configured and arranged to provide electrical stimulation signals to an electrical stimulation lead coupled to the implantable control module for stimulation of patient tissue. The system also includes an external programming unit configured and arranged to communicate with the processor of the implantable control module using the antenna and to provide or update stimulation parameters for production of the electrical stimulation signals, wherein the first external programming unit is configured and arranged to communicate with a database remote from the external programming unit and the implantable control module to store the stimulation parameters at the database for use by other users.

In at least some embodiments, the external programming unit includes a user interface configured and arranged to receive input from a user, and a processor in communication with the user interface and configured and arranged to perform the following actions: a) requesting authorization from the patient for use of patient data, b) receiving authorization from the patient, and c) in response to the patient authorization, sending patient data to the database. This external programming unit can be used in any of the embodiments described herein.

In at least some embodiments, the system also includes the database remote from the external programming unit and the implantable control module and configured and arranged to store the stimulation parameters. In at least some embodiments, the database contains medical image data as part of the aggregated data. This database can be used in any of the embodiments described herein.

In at least some embodiments, the database includes a user interface configured and arranged to receive input from a user, and a processor in communication with the user interface and configured and arranged to perform the following actions: a) requesting authorization patient for use of patient data, b) receiving authorization from the patient, c) receiving patient data, and d) in response to the patient authorization, aggregating the patient data with other data at the database. This database can be used in any of the embodiments described herein.

In at least some embodiments, the database includes a user interface configured and arranged to receive input from a user, and a processor in communication with the user interface and configured and arranged to perform the following actions: a) receiving a patient pain condition, b) correlating the patient pain condition with aggregated data from a plurality of patients and a plurality of pain conditions, and c) presenting recommendations for one or more stimulation parameters. In at least some embodiments, correlating the patient pain condition with the aggregated data includes correlating the patient pain condition with a previously prepared correlation construct generated using the aggregated data. This database can be used in any of the embodiments described herein.

Yet another embodiment is a non-transitory computer-readable medium having processor-executable instructions for providing stimulation recommendations based on aggregated data for a plurality of patients and a plurality of pain conditions, the processor-executable instructions when installed onto a device enable the device to perform actions, including: receiving a patient pain condition; correlating patient pain condition with aggregated data; and presenting recommendations for one or more stimulation parameters.

In at least some embodiments, the processor-executable instructions when installed onto a device enable the device to perform the following additional actions: requesting authorization patient for use of patient data; receiving authorization from the patient; receiving patient data; and in response to the patient authorization, aggregating the patient data with other data at the database.

In at least some embodiments, the processor-executable instructions when installed onto a device enable the device to perform the following additional action: generating a target stimulation map from the aggregated data.

In at least some embodiments, the processor-executable instructions when installed onto a device enable the device to perform the following additional action: receiving at least one medical image of the patient.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic block diagram of one embodiment of an electrical stimulation system, according to the invention;

FIG. 2 is a schematic block diagram of another embodiment of an electrical stimulation system, according to the invention;

FIG. 3 is a schematic block diagram of another embodiment of an electrical stimulation system, according to the invention;

FIG. 4 is a schematic block diagram of one embodiment of a patient interface unit, according to the invention;

FIG. 5 is a schematic block diagram of one embodiment of an external programming unit, according to the invention;

FIG. 6 is a schematic block diagram of one embodiment of a database, according to the invention;

FIG. 7 is a flowchart of one embodiment of a method for adjusting stimulation parameters, according to the invention;

FIG. 8 is a flowchart of one embodiment of a method for testing a range of stimulation parameters, according to the invention;

FIG. 9 is a flowchart of one embodiment of a method for requesting patient authorization for using patient data, according to the invention;

FIG. 10 is a flowchart of another embodiment of a method for obtaining stimulation recommendations, according to the invention;

FIG. 11 is a schematic side view of one embodiment of a lead and control module, according to the invention;

FIG. 12 is a schematic side view of another embodiment of a lead and control module, according to the invention;

FIG. 13A is a schematic side view of one embodiment of an implantable control module configured and arranged to electrically couple to an elongated device, according to the invention; and

FIG. 13B is a schematic side view of one embodiment of a lead extension configured and arranged to electrically couple an elongated device to an implantable control module, according to the invention.

### DETAILED DESCRIPTION

The present invention is directed to the area of implantable electrical stimulation systems and methods of making and using the systems. The present invention is also directed to implantable electrical stimulation systems include devices or methods for providing feedback regarding electrical stimulation, as well as methods of making and using the electrical stimulation systems.

Suitable implantable electrical stimulation systems include, but are not limited to, a least one lead with one or more electrodes disposed along a distal end of the lead and one or more terminals disposed along the one or more proximal ends of the lead. Leads include, for example, percutaneous leads, paddle leads, and cuff leads. Examples of electrical stimulation systems with leads are found in for example, U.S. Pat. Nos. 6.181.969; 6,516,227; 6,609,029; 6,609,032; 6,741,892; 7.949.395; 7,244,150; 7,672.734; 7,761,165; 7,974,706; 8,175,710; 8,224,450; and 8,364,278; and U.S. Patent Application Publication No. 2007/0150036, all of which are incorporated by reference.

An electrical stimulation system can include a local or non-local network of devices that can be used in a session to program, visualize or interact during a medical office procedure, such as a session to program a neurostimulator. In at least some embodiments, the system allows a patient to provide feedback on the effects of the therapy while the clinician or programmer adjusts the parameters of the medical device.

For example, the clinician or programmer uses an external programming unit that communicates (directly or through a secondary device) with an implantable control module that delivers therapy or stimuli to a patient. The clinician or programmer perceives or receives from the patient feedback regarding the clinical effects of the stimuli. The feedback can then be recorded into the external programming unit or other components of the system (locally or remotely). This feedback is utilized to define a clinical effects map that correlates therapy delivered with the clinical effects experienced. Alternatively or additionally, both the programmer and the patient may input the clinical effects into the system. The patient may provide feedback to the programming unit via a patient interface unit. Patient feedback may also be recorded as bio-signals from the patient (EEG, EVIG, CMAP, ECG, skin resistance, muscle tone, movement, vibration, rigidity, temperature, breathing, oxygen levels, chemical concentrations, skin resistance, gait, skin tone, force, pressure, and the like.)

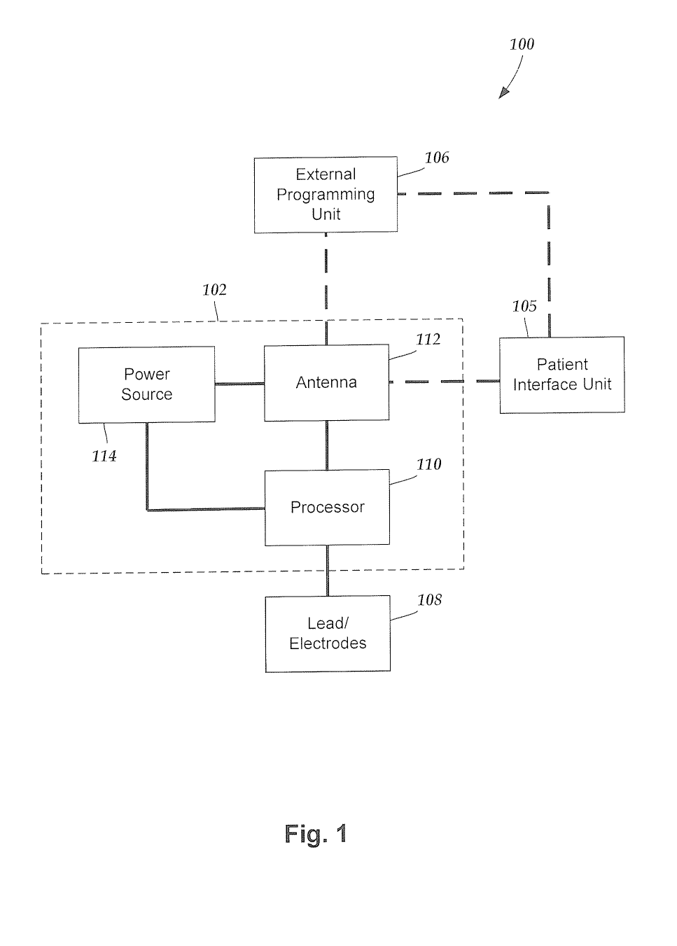

FIG. 1 illustrates schematically one embodiment of an electrical stimulation system 100 that includes an implantable control module (e.g., an implantable electrical stimulator or implantable pulse generator) 102, one or more leads 108 with electrodes, one or more external programming units 106, and a patient interface unit 105. Alternatively, the implantable control module 102 can be part of a microstimulator with the electrodes disposed on the housing of the microstimulator. The microstimulator may not include a lead or, in other embodiments, a lead may extend from the microstimulator. It will be understood that the electrical stimulation system can include more, fewer, or different components and can have a variety of different configurations including those configurations disclosed in the references cited herein. For example, although FIG. 1 illustrates one external programming units 106, one control module 102, and one patient interface unit 105, it will be understood that the system can include more than one external programming unit, more than one control module, and more than one patient interface unit.

The lead 108 is coupled, or coupleable, to the implantable control module 102. The implantable control module 102 includes a processor 110, an antenna 112 (or other communications arrangement), a power source 114, and a memory 116, as illustrated in FIG. 1.

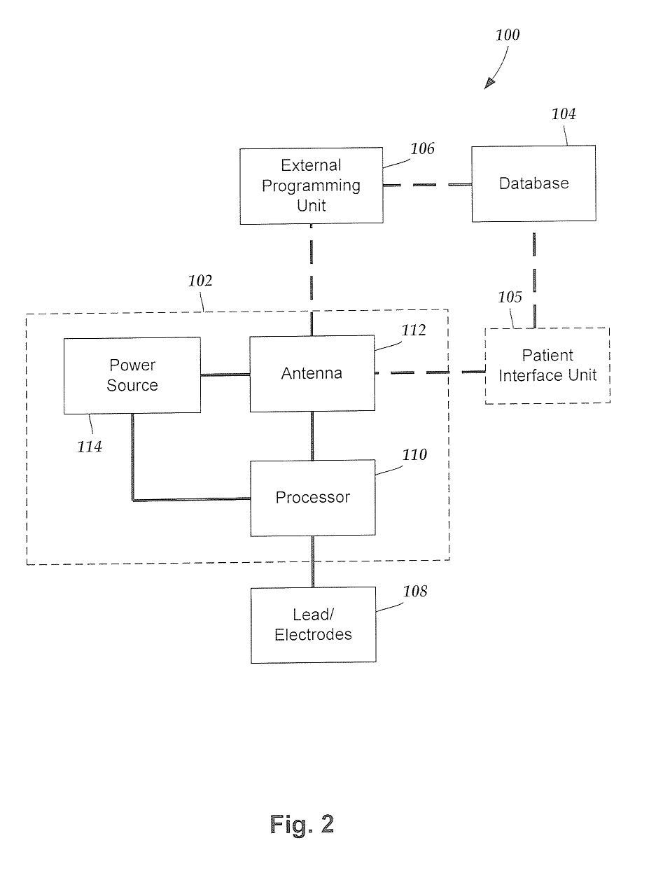

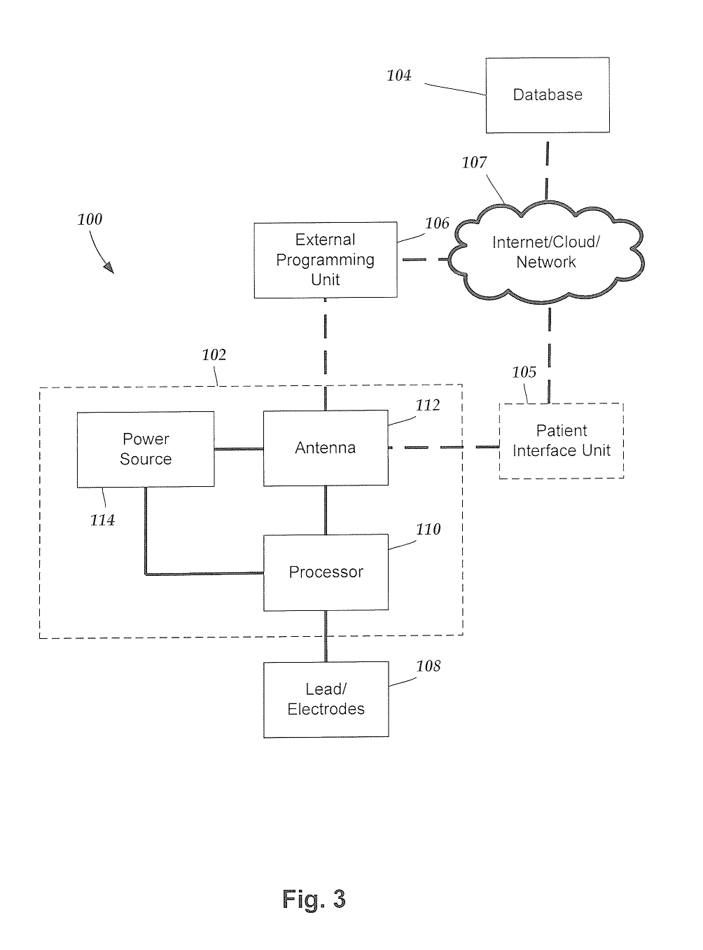

FIG. 2 illustrates another embodiment that includes a database 104 with communicates with the external programming unit 106 and optional patient interface unit 105. FIG. 3 illustrates another embodiment in which the external programming unit 106 and optional patient interface unit 105 communicate through the Internet, a cloud, or a local or wide area network 107 (including wireless local or wide area networks) or any combination thereof, or any other suitable intermediary or combination of intermediaries, to the database 104.

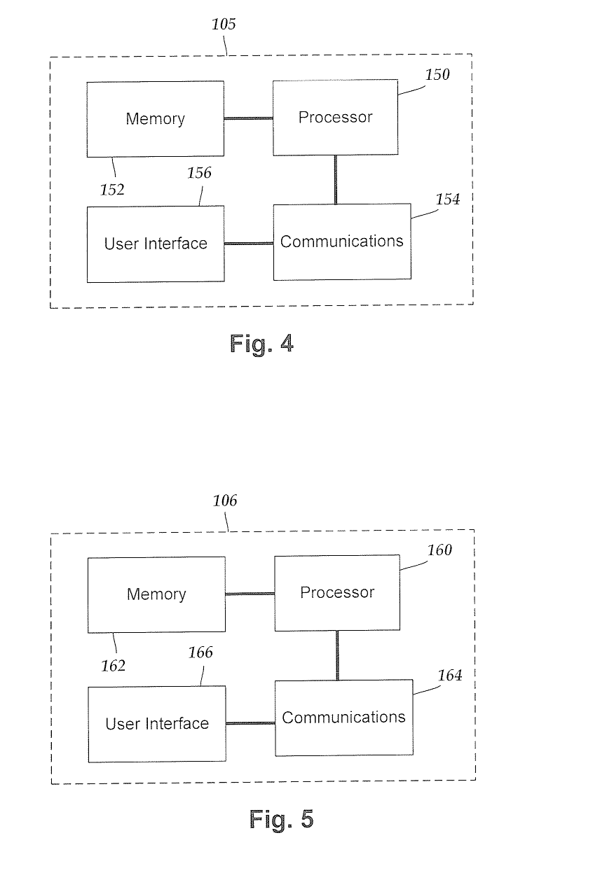

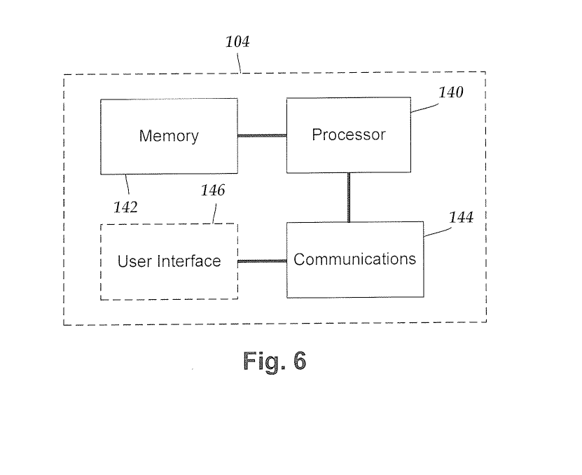

One example of a patient interface unit 105 is illustrated in FIG. 4 and includes a processor 150, a memory 152, a communications arrangement 154 (such as an antenna or any other suitable communications device such as those described below), and an optional user interface 156. Suitable devices for use as a patient interface unit can include, but are not limited to, a computer, a tablet, a mobile telephone, a personal desk assistant, a dedicated device for external programming, remote control, or the like. In at least some embodiments, the patient interface unit 105 can also be an external programming unit.

One example of an external programming unit 106 is illustrated in FIG. 5 and includes a processor 160, a memory 162, a communications arrangement 164 (such as an antenna or any other suitable communications device such as those described below), and a user interface 166. Suitable devices for use as an external programming unit can include, but are not limited to, a computer, a tablet, a mobile telephone, a personal desk assistant, a dedicated device for external programming, remote control, or the like. It will be understood that the external programming unit 106 and patient interface unit 105 can include a power supply or receive power from an external source or any combination thereof. In at least some embodiments, the external programming unit 106 may also be a patient interface unit.

One example of a database 104 is illustrated in FIG. 4 and includes a processor 140 a memory 142, a communications arrangement 144 (such as an antenna or any other suitable communications device such as those described below), and an optional user interface 146. Suitable devices for use as a remote data storage unit can include, but are not limited to, a computer, a tablet, a server or server farm, a dedicated device for data storage, a hard drive, cloud storage arrangement, or the like. It will be understood that the external programming unit 106 and remote data storage unit 104 can include a power supply or receive power from an external source or any combination thereof. In some embodiments, the database 104 can also act as a remote storage unit for storage and retrieval of stimulation parameters and other stimulation data. Examples of remote storage units and their use and operation in electrical stimulation systems can be found in U.S. Provisional patent application Ser. No. ______, entitled “Systems and Methods for Synchronizing Stimulation Data”, filed Jul. 24, 2014 (Attorney Docket No. BSNC-1-373.0), incorporated herein by reference.

Methods of communication between devices or components of a system can include wired or wireless (e.g., RF, optical, infrared, near field communication (NFC), Bluetooth™, or the like) communications methods or any combination thereof. By way of further example, communication methods can be performed using any type of communication media or any combination of communication media including, but not limited to, wired media such as twisted pair, coaxial cable, fiber optics, wave guides, and other wired media and wireless media such as acoustic, RI optical, infrared, NEV, Bluetooth™ and other wireless media. These communication media can be used for communications units 144, 154, 164 or as antenna 112 or as an alternative or supplement to antenna 112.

Turning to the control module 102, some of the components (for example, a power source 114, an antenna 112, and a processor 110) of the electrical stimulation system can be positioned on one or more circuit boards or similar carriers within a sealed housing of the control module (implantable pulse generator,) if desired. Any power source 114 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Pat. No. 7,437,193, incorporated herein by reference.

As another alternative, power can be supplied by an external power source through inductive coupling via the antenna 112 or a secondary antenna. The external power source can be in a device that is mounted on the skin of the user or in a unit that is provided near the user on a permanent or periodic basis.

If the power source 114 is a rechargeable battery, the battery may be recharged using the antenna 112, if desired. Power can be provided to the battery for recharging by inductively coupling the battery through the antenna to a recharging unit external to the user.

A stimulation signal, such as electrical current in the form of electrical pulses, is emitted by the electrodes of the lead 108 (or a microstimulator) to stimulate neurons, nerve fibers, muscle fibers, or other body tissues near the electrical stimulation system. Examples of leads are described in more detail below. The processor 110 is generally included to control the timing and electrical characteristics of the electrical stimulation system. For example, the processor 110 can, if desired, control one or more of the timing, frequency, strength, duration, and waveform of the pulses. In addition, the processor 110 can select which electrodes can be used to provide stimulation, if desired. In some embodiments, the processor 110 selects which electrode(s) are cathodes and which electrode(s) are anodes. In some embodiments, the processor 110 is used to identify which electrodes provide the most useful stimulation of the desired tissue.

With respect to the control module 102, patient interface unit 105, external programming unit 106, and database unit 104, any suitable processor 110, 140, 150, 160 can be used in these devices. For the control module 102, the processor 110 is capable of receiving and interpreting instructions from an external programming unit 106 that, for example, allows modification of pulse characteristics. In the illustrated embodiment, the processor 110 is coupled to the antenna 112. This allows the processor 110 to receive instructions from the external programming unit 106 to, for example, direct the pulse characteristics and the selection of electrodes, if desired. The antenna 112, or any other antenna described herein, can have any suitable configuration including, but not limited to, a coil, looped, or loopless configuration, or the like.

In one embodiment, the antenna 112 is capable of receiving signals (e.g., Rh signals) from the external programming unit 106. The external programming unit 106 can be a home station or unit at a clinician's office or any other suitable device. In some embodiments, the external programming unit 106 can be a device that is worn on the skin of the user or can be carried by the user and can have a form similar to a pager, cellular phone, or remote control, if desired. The external programming unit 106 can be any unit that can provide information to the control module 102. One example of a suitable external programming unit 106 is a computer operated by the user or clinician to send signals to the control module 102. Another example is a mobile device or an application on a mobile device that can send signals to the control module 102

The signals sent to the processor 110 via the antenna 112 can be used to modify or otherwise direct the operation of the electrical stimulation system. For example, the signals may be used to modify the pulses of the electrical stimulation system such as modifying one or more of pulse duration, pulse frequency, pulse waveform, and pulse strength. The signals may also direct the control module 102 to cease operation, to start operation, to start charging the battery, or to stop charging the battery.

Optionally, the control module 102 may include a transmitter (not shown) coupled to the processor 110 and the antenna 112 for transmitting signals back to the external programming unit 106 or another unit capable of receiving the signals. For example, the control module 102 may transmit signals indicating whether the control module 102 is operating properly or not or indicating when the battery needs to be charged or the level of charge remaining in the battery. The processor 110 may also be capable of transmitting information about the pulse characteristics so that a user or clinician can determine or verify the characteristics.

Any suitable memory 116, 142, 152, 162 can be used for the respective components of the system 100. The memory 116, 142, 152, 162 illustrates a type of computer-readable media, namely computer-readable storage media. Computer-readable storage media may include, but is not limited to, nonvolatile, removable, and non-removable media implemented in any method or technology for storage of information, such as computer readable instructions, data structures, program modules, or other data. Examples of computer-readable storage media include RAM. ROM, EEPROM, flash memory, or other memory technology, CD-ROM, digital versatile disks (“DVD”) or other optical storage, magnetic cassettes, magnetic tape, magnetic disk storage or other magnetic storage devices, or any other medium which can be used to store the desired information and which can be accessed by a computing device.

Communication methods provide another type of computer readable media; namely communication media. Communication media typically embodies computer-readable instructions, data structures, program modules, or other data in a modulated data signal such as a carrier wave, data signal, or other transport mechanism and include any information delivery media. The terms “modulated data signal,” and “carrier-wave signal” includes a signal that has one or more of its characteristics set or changed in such a manner as to encode information, instructions, data, and the like, in the signal. By way of example, communication media includes wired media such as twisted pair, coaxial cable, fiber optics, wave guides, and other wired media and wireless media such as acoustic, RF, infrared, and other wireless media.

The user interfaces 156, 166 of the external programming unit 106 and patient interface unit 105 and optional user interface 146 of the database 104 can be, for example, a keyboard, mouse, touch screen, track ball, joystick, voice recognition system, or any combination thereof, and the like. Alternatively or additionally, the user interfaces 156, 166 of the external programming unit 106 and of the patient interface unit 105 can include one or more microphones, sensors, cameras, or the like to obtain patient feedback. For example, the patient may provide feedback verbally (e.g. voice command recognition, voice recordings) or visually (e.g. video of patient, non-touch gesture recognition, or the like). In at least some embodiments, at least a portion (or even all of) the patient feedback may be recorded as bio-signals from the patient (EEG, EMG, CMAP, ECG, skin resistance, muscle tone, movement, vibration, rigidity, temperature, breathing, oxygen levels, chemical concentrations, skin resistance, gait, skin tone, force, pressure, or the like.) In at least some embodiments, such as those illustrated in FIGS. 2 and 3 without the optional patient interface unit, patient feedback can be provided by the clinician or other user through the external programming unit 106.

In at least some embodiments, the patient interface unit 105 may also have controls to adjust parameters of the stimulation, which are optionally limited to an acceptable range by the clinician (using the patient interface unit or external programming unit) or by default settings of the electrical stimulation system or control module or any other suitable mechanism. Such parameters can include, for example, one or more of stimulation amplitude range, stimulation frequency range, stimulation pulse width, stimulation duration, interval between delivery of stimulation, electrode combinations, selection of anode(s) and cathode(s), or the like or any combination thereof.

In at least some embodiments, as the person delivering therapy adjusts stimulation parameters using the external programming unit 106, the patient can then use the patient interface unit 105 to identify the effects of the stimulation parameters. For example, the patient can indicate in which areas of the body there is perception of stimulation (e.g. paresthesia) or indicate a pain level or indicate a score for satisfaction with treatment, or any combination thereof. In at least some embodiments, the patient interface unit 105 may also allow the patient to input areas where the patient needs or desires more stimulation and areas where the patient needs or desires less stimulation (for example, areas with side effects). This information can be provided to the external programming unit 106 for the programmer to adjust stimulation parameters.

In at least some embodiments, the system utilizes a figure of merit of the adequacy, or a rating, of the stimulation provided. The figure of merit can be based on, for example, one or more of the following: therapeutic effect, patient satisfaction, level or presence of side effects, patient dissatisfaction, or the like or any combination thereof. The figure of merit can be, for example, a numerical value, an alphanumerical rating, a descriptive rating, or any other suitable rating mechanism or any combination thereof. The figure of merit may be provided by the clinician, the patient, or both and can be input through the external programming unit 106, patient interface unit 105, or other device or any combination of these or other devices. In at least some embodiments, the figure of merit can be used to enhance, adjust, or guide stimulation delivery.

The figure of merit may be determined from multiple ratings provided by the clinician, the patient, or both. In at least some embodiments, the determination of a figure of merit may employ different weights to different parameters in the calculation. For example, the determination may add more value to certain preferred clinical outcomes, may add more negative value to particular side effects, may add less negative value to power consumption, or the like. The weights and parameters entering the figure of merit may be defined by the users of the system (e.g. patient, clinician, programmer, or any combination thereof) or may be pre-set by the system. The system may allow different users to define different figures of merit that can be optionally shared via the system.

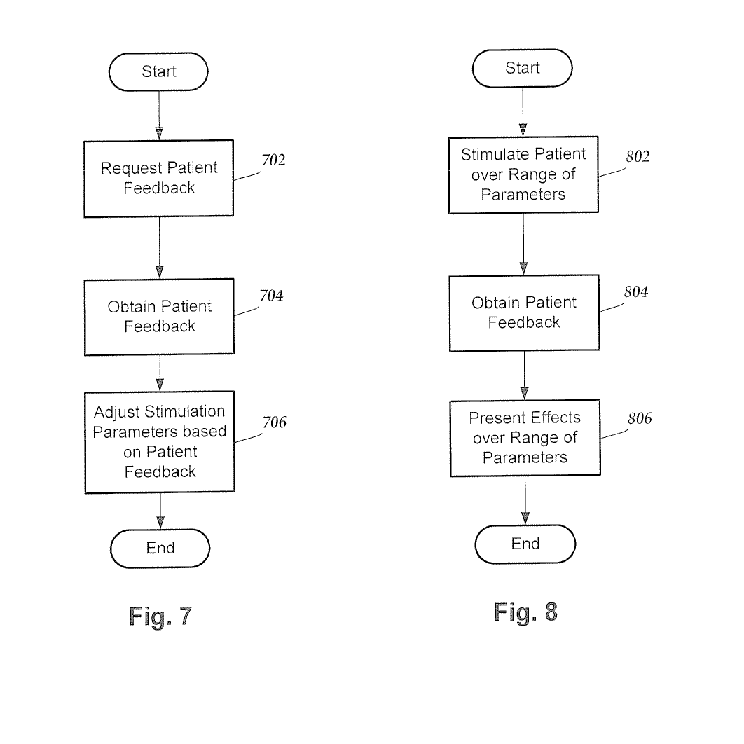

FIG. 7 is a flowchart of one embodiment of a method of adjusting stimulation parameters. In step 702, patient feedback is requested. The request can be made on the external programming unit, patient interface unit, or using any other suitable device or method, or any combination thereof. In step 704, the patient feedback is obtained. The patient may provide the feedback directly to the system through, for example, the patient interface unit or may provide the feedback to the clinician who inputs the feedback through, for example, the external programming unit. In step 706, the system analyzes the feedback and provides recommendations for adjusting the stimulation parameters based on the patient feedback. In some embodiments, the analysis, or portions of the analysis, may be performed by the external programming unit or another local device. In some embodiments, the analysis, or portions of the analysis, can be performed using a remote device.

In at least some embodiments, the system may allow creation of different therapeutic options based, for example, on different figures of merit or situations for the patient. Each therapeutic option may have one or more different stimulation parameters from other therapeutic options. For example, the system may provide or allow different stimulation programs for one or more of the following conditions or situations: sleep, walking, condition exacerbation, acute episodes, preventative therapies, activity based, and the like. In at least some embodiments, the patient user interface 105 or the external programming unit 106 may be designed to create multiple areas of stimulation and multiple programs to target different areas.

In at least some embodiments, the stimulation controls of the external programming unit 106 or, optionally, the patient interface unit 105 may be designed to allow changing the shape of the stimulation area, size of the stimulation area, strength of the stimulation, perception pattern (for example, type of sensation or no sensation) of the stimulation, or the like, or any combination thereof. In at least some embodiments, the external programming unit 106 or, optionally, the patient interface unit 105 may allow a user to move the stimulation to different anatomical areas (for example, using directional controls, such as arrows, or a joystick or mouse or other mechanism) or by marking areas in a map representing the body. In at least some embodiments, the external programming unit 106 or, optionally, the patient interface unit 105 may allow a use to increment or decrement stimulation in certain areas of the body or to add or remove stimulation from certain areas of the body. In at least some embodiments, the external programming unit 106 or, optionally, the patient interface unit 105 may allow a user (for example, a clinician or a patient) to increase or decrease the intensity of stimulation in areas of the body. In at least some embodiments, these areas can be mapped by the patient using the patient interface unit 105.

In at least some embodiments, a human figure, or parts of human anatomy, can be depicted in 2-dimensional and/or 3-dimensional representations on the patient user interface unit 105 or the external programming unit 106 to allow the user (for example, a patient or a clinician) to input areas of the body that, for example, experience stimulation-induced effects (for example, where stimulation is perceived or where stimulation is evidenced), experience pain, are desired stimulation areas, are areas where stimulation is not desired, are areas where side effects occur, or the like or any combination thereof. In at least some embodiments, the areas of the body can be marked based on level of intensity of stimulation, pain, or side effects or any other suitable parameter. In at least some embodiments, the user can indicate different levels of intensity, for example, areas of intense pain or mild pain, or areas of intense stimulation or mild stimulation). It will be recognized that more than two different levels of intensity can be represented. In addition, in at least some embodiments, aspects of the reported pain, stimulation, or side effects (or any combination thereof) may be recorded in addition to the intensities of said effects. For example, pain quality (for example, tingling, burning, stabbing, shooting, throbbing, or the like), stimulation quality (for example, tingling, buzzing, hot, cold, cool, warm, or the like), other characteristics (for example, worst pain, overall pain, pain that is ‘superficial’ or ‘deep’, stimulation effects which are ‘superficial’ or ‘deep’, or the like), or any combination thereof can be recorded.

FIG. 8 is a flowchart of one embodiment of a method of testing a range of stimulation parameters. In step 802, the patient is stimulated over a range of stimulation parameters. In some embodiments, only a single stimulation parameter (for example, stimulation amplitude, stimulation duration, selection of electrodes) is varied over a range. In other embodiments, more than one stimulation parameter is varied. In step 804, the patient feedback is obtained. The patient may provide the feedback directly to the system through, for example, the patient interface unit or may provide the feedback to the clinician who inputs the feedback through, for example, the external programming unit. In step 806, the system analyzes the feedback and presents the clinical effects (for example, patient feedback, stimulation efficacy, presence of side effects, strength of side effects, or any combination thereof) for the range of stimulation parameters. In some embodiments, the system may also provide recommendations for the stimulation parameters based on the patient feedback. In some embodiments, the analysis, or portions of the analysis, may be performed by the external programming unit or another local device. In some embodiments, the analysis, or portions of the analysis, can be performed using a remote device.

In at least some embodiments, the system stores the different therapeutic options, clinical effects map, figures of merit of the particular patient in one or more of the control module 102, the external programming unit 106, patient user interface 105, or remote database 104. In at least some embodiments, the clinical effects observed by the clinician or reported by the patient are stored in relation to the particular set of parameters associated therewith. The parameters of the stimulation can include the stimulation parameters at the time of feedback or may also include historical information, such as the parameters used for stimulation delivered over a period of time.

In at least some embodiments, the external programming; unit 106, control module 102, or patient interface unit 105 (or any combination thereof) may be configured to adjust the stimulation parameters in a selected pattern either automatically or semi-automatically (with some user control over selection of which parameters to test or sets of initial parameters) to create a map of clinical effects over a related set of stimulation conditions. For example, an algorithm may guide several sets of parameters and stimulation combinations in a given order and record clinical effects as the different parameter sets are exercised. As another example, an algorithm can adjust one or more of amplitude, frequency, duration, electrode selection, or the like or any combination thereof to obtain a map of clinical effects for varied stimulation parameters. In at least some embodiments, the patient, user, or system can provide information, ratings, or figures of merit for the different sets of stimulation parameters.

The system can also incorporate demographics (for example, age, gender, ethnicity, height, weight, or the like) and disease-specific details (for example, pain etiology(ies), number of prior back surgeries, relevant diagnoses, imaging findings, or the like) in the information provided to the external programming unit 106, control module 102, or patient interface unit 105 (or any combination thereof).

In at least some embodiments, the system may also be capable of receiving medical imaging information (for example, photographs, video, MRI images, CT scans, ultrasound images, anatomical atlases, or the like) and device information. The system may be configured to allow a user to upload MRI scans, CT scans, ultrasound images, or other visualization information of the anatomical targets for stimulation. In at least some embodiments, the system or a user may also correlate this visualization information to the position of the lead and electrical contacts to aid in the recommendation of stimulation targets and electrode combinations.

In at least some embodiments, information obtained from a programming or patient interactive session can be recorded and stored on one or more of the control module 102, the external programming unit 106, patient user interface 105, or remote database 104. This information may be used to provide additional statistical data to correlate stimulation parameters, targets of stimulation, anatomical placements, lead configurations, diagnostics, symptoms, patient characteristics, or the like. This information may also be used to inform a statistical-based algorithm that aggregates the information and calculates potential or recommend lead or electrode configurations for the patient. Such configurations, the underlying information, or portions of the information may be made available to other programmers, clinicians, or patients.

In at least some embodiments, the system may use information (for example, stimulation parameters, patient or clinician feedback, figures of merit, ratings, or the like) inputted to one or more of the control module 102, the external programming unit 106, patient user interface 105, or remote database 104 to calculate derived metrics that can be used by the programmer to assess programming effectiveness. Examples include stimulation effectiveness, pain/paresthesia concordance, worst pain/paresthesia concordance, quantification of the degree to which the pain that has ‘neuropathic’ descriptors assigned to it by the patient is covered with stimulation, quantification of the degree to which pain that has ‘nociceptive’ or ‘pain’ descriptors assigned to it by the patient is covered with stimulation, side effect correlation to parameters or location, or the like.

In at least some embodiments, the system can use the patient- or clinician-provided feedback to automatically adjust stimulation parameters. For example, if the patient or clinician marks an area as too intense the system may automatically reduce the stimulation amplitude in that area (by changing electrode combinations or electrical parameters of the electrodes), or it could recommend a next settings of parameters which the user can accept or migrate to. In at least some embodiments, the system can determine or present a “target volume of activation” and the user may adjust stimulation parameters to overlap the target.

In at least some embodiments, the system contains information regarding targets based on historical and statistical data within a single patient or across many patients. For example, a depiction of a human and selection of areas of pain can produce a pain map that can be utilized to input areas of pain (or other symptoms) by the patient or clinician or both. In at least some embodiments, the system can use this pain map to provide recommended target volumes of activation (where electric current should be delivered) or recommended initial stimulation parameters or recommended stimulation parameter ranges for testing. This information can be used to plan for the surgical procedure to implant the control module and lead(s) and programming of the control module.

In at least some embodiments, the feedback provided or perceived from the patient or clinician is mapped in the patient interface unit 105, external programming unit 106, or database 104 or any combination thereof and can be available to be used by the system to provide feedback to the user to aid in the stimulation adjustment. In at least some embodiments, the system may use the information to provide a recommended next step for adjustment of stimulation parameters or other adjustment to therapeutic treatment. In at least some embodiments, the system may use the information and aggregate it with information from the same or other patients to create a statistical estimation of clinical effects in relation to stimulation parameters, user characteristics, or any combination thereof.

In at least some embodiments, the system transmits data collected from the patient (for example, clinical effects, patient or clinician feedback, stimulation parameters, or any combination thereof) to be transmitted to the database 104. The information in the database 104 may be used for aggregation and recommendation of stimulation adjustments at a different clinical session or may be provided to the patient or clinician to download into his/her devices (e.g., control module 102, the external programming unit 106, patient user interface 105, personal devices or database, or the like or any combination thereof).

For data aggregation, the user (for example, clinician, patient, or other user) may choose to aggregate a group of data (for example, data from a particular patient set to a given medical center or centers) or may choose to aggregate data from a larger group of patients. Data aggregation can be executed from a local system (relative to the clinician, patient, site of surgery, or the like) or may be done remotely (e.g. remote database and cloud computing).

In at least some embodiments, the system has a feature for the patient to authorize the information to be used in the aggregation of data or exported for further analysis. For example, the patient may produce an electronic signature, paper signature that is recorded, video or audio authorizing the aggregation, or the like. If the patient authorizes data aggregation, the patient's data can be used for analysis by the system to inform programming or stimulation delivery to other patients or to analyze further. If the patient does not authorize the information to be used, the system will exclude the patient's data from the aggregation of data from other patients. In at least some embodiments, the system excludes patient identification data for aggregation, export or analysis.

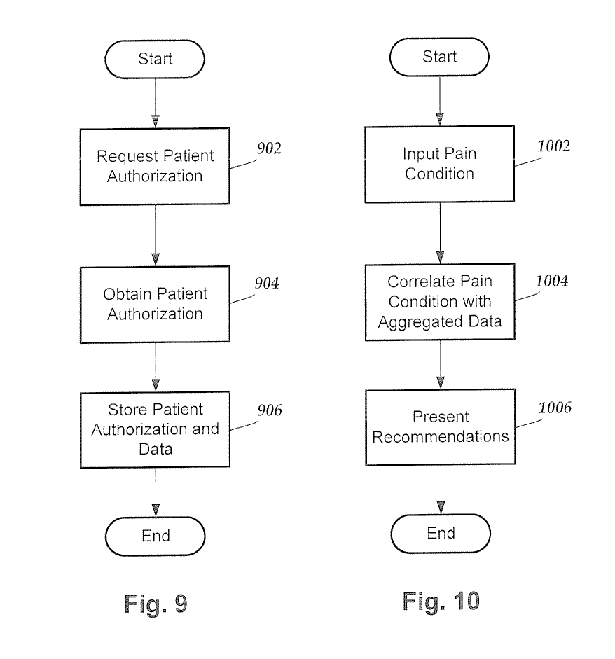

FIG. 9 is a flowchart of one embodiment of a method of obtaining patient authorization. In step 902, the system requests patient authorization. The request can be made through the clinician, through the external programming unit, or the patient interface unit or any other suitable mechanism. In step 904, the patient authorization is obtained. In at least some embodiments, that authorization is in the form of an electronic signature, paper signature that is recorded, video or audio authorizing the aggregation, or the like. In step 906, the patient authorization and authorized patient data is submitted to the database. This submission of authorization and data may be performed simultaneously or performed at different times. If the data has been previously submitted to the database, the patient's authorization may be used to allow the previously submitted data to be aggregated with other data.

In at least some embodiments, the system may also allow experienced programmers of the stimulation system to propose stimulation parameter or lead configurations which can be downloaded by other users of the system.

In at least some embodiments, multiple external programming units 106, patient interface units 105, or any combination thereof can be connected either locally or remotely into a network (local or wide area or virtual) allowing multiple programmers or users at the same time. In at least some embodiments, when multiple programmers are using a system, the system can be configured to allow several programmers to work at the same time or at different times. In at least some embodiments, the system can also be configured to allow different priorities between users. In at least some embodiments, the system can also be configured to allow particular functions to take precedence regardless of who is the user (for example, an emergency stimulation shut down command).

In at least some embodiments, the aggregation of data can include the creation of target stimulation maps for particular pain conditions. The system can aggregate effective stimulation parameters for many patients who experience pain due to different conditions and different locations in the body. The system (automatically, semi-automatically, or with clinician input) can evaluate, based on figures of merit or other feedback or data from each of the patients, and provide a statistical target area to stimulate in the spinal cord of a patient to ensure maximum likelihood of success, based on the figure of merit. In at least some embodiments, a clinician can then input the pain condition of the patient and read from the target stimulation map the recommended stimulation locations or parameters or both and a statistical distribution of results. In at least some embodiments, the system can provide a visual anatomical representation of the target area (for example, as a single point, a surface, an anatomical structure, or a volume). The target of activation may also be determined via functional outcomes of stimulation (e.g. one vertebral space above area where toe flexion occurs during stimulation). In at least some embodiments, this information can be used to plan the placement of the electrodes. In at least some embodiments, once the electrodes are in place, the location of the electrodes relative to the spinal cord can be input to the system. In at least some embodiments, the system can then recommend one or more sets of stimulation parameters or parameter ranges to place stimulation in the target area.

In at least some embodiments, the target area may be represented in the system and a volume of activation based on current stimulation parameters to visually indicate the amount of overlap (and non-overlap) of target volume and volume of activation. In at least some embodiments, the system may provide surface or volumetric measures of overlap and non-overlap. The user can use the stimulation controls to place stimulation in the target or as close as adequate. This is particularly useful for indications in which patients may not feel the stimulation or in which the effects of stimulation take too long to be measured during a programming session. In at least some embodiments, the user can also use patient feedback to set the target of stimulation.

FIG. 10 is a flowchart of one embodiment of a method of obtaining recommendations for stimulation sites, parameters, or any combination therefrom from the system. In step 1002, the pain condition is input into the system using, for example, the external programming unit, the patient interface unit, or any other input device. The pain condition can be expressed in any suitable manner that is accepted by the system, such as, for example, descriptive text, selection from a list of pain sites or symptoms or other indicators, identification of pain regions on an anatomical representation, or any other suitable method of indicating the source, symptom, or site of the pain. In step 1004, the system correlates the information provided on the pain condition with the aggregated data. Such correlation may be made with the data directly or with a previously prepared correlation construct, such as a target stimulation map, pain map, pain concordance, or the like, produced using the aggregated data. In step 1006, recommendations for stimulation are provided by the system to the clinician, patient, or other user. These recommendations can include one or more of the following: stimulation site, initial stimulation parameters, stimulation parameter ranges, or any combination thereof.

It will be understood that the system can include one or more of the methods described hereinabove with respect to FIGS. 7-10 in any combination. The methods, systems, and units described herein may be embodied in many different forms and should not be construed as limited to the embodiments set forth herein. Accordingly, the methods, systems, and units described herein may take the form of an entirely hardware embodiment, an entirely software embodiment or an embodiment combining software and hardware aspects. The methods described herein can be performed using any type of processor or any combination of processors where each processor performs at least part of the process.

It will be understood that each block of the flowchart illustrations, and combinations of blocks in the flowchart illustrations and methods disclosed herein, can be implemented by computer program instructions. These program instructions may be provided to a processor to produce a machine, such that the instructions, which execute on the processor, create means for implementing the actions specified in the flowchart block or blocks or described for the control modules, external programming units, remote data storage units, systems and methods disclosed herein. The computer program instructions may be executed by a processor to cause a series of operational steps to be performed by the processor to produce a computer implemented process. The computer program instructions may also cause at least some of the operational steps to be performed in parallel. Moreover, some of the steps may also be performed across more than one processor, such as might arise in a multi-processor computer system. In addition, one or more processes may also be performed concurrently with other processes, or even in a different sequence than illustrated without departing from the scope or spirit of the invention.

The computer program instructions can be stored on any suitable computer-readable medium including, but not limited to, RAM, ROM, EEPROM, flash memory or other memory technology, CD-ROM, digital versatile disks (“DVD”) or other optical storage, magnetic cassettes, magnetic tape, magnetic disk storage or other magnetic storage devices, or any other medium which can be used to store the desired information and which can be accessed by a computing device.

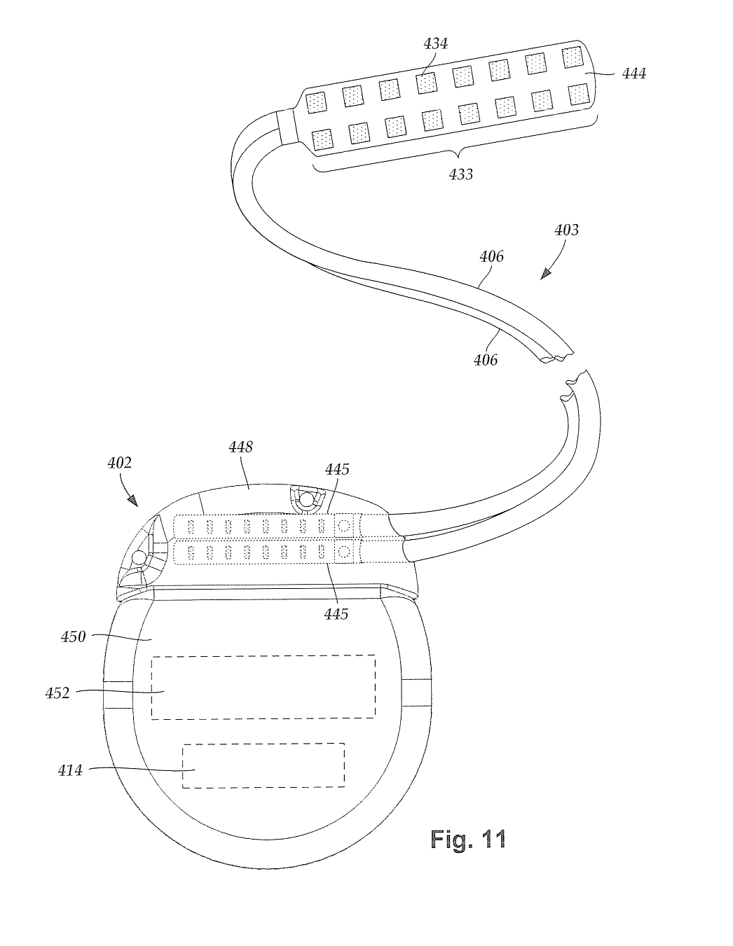

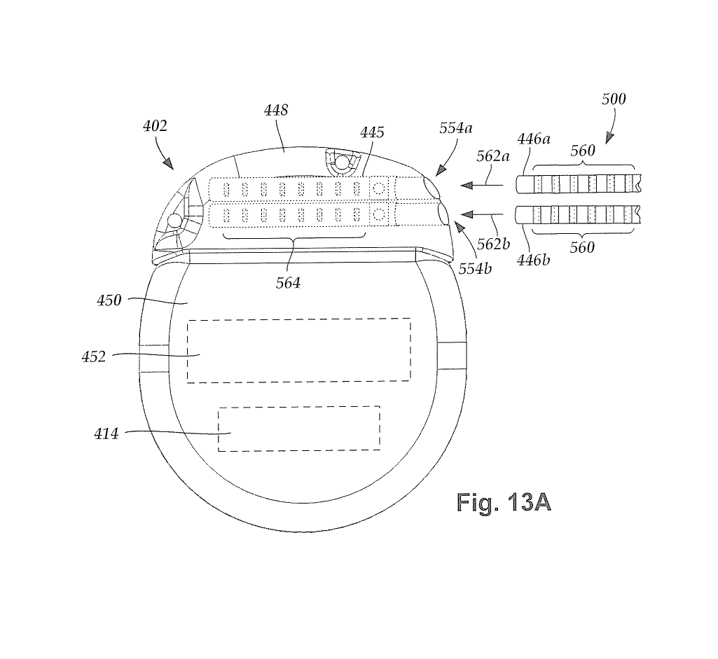

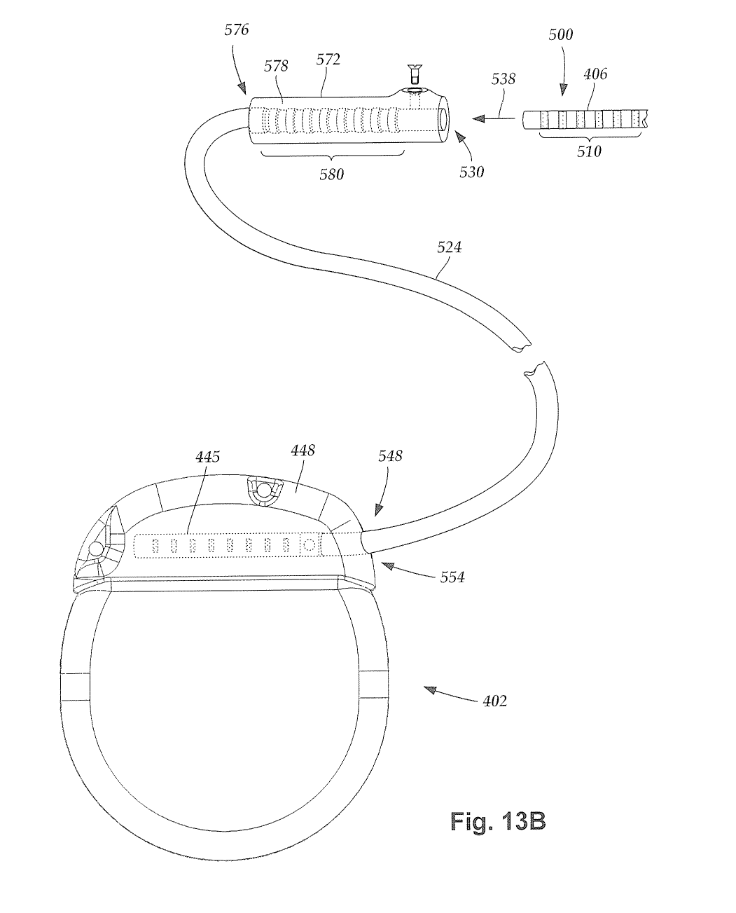

FIG. 11 illustrates one embodiment of a control module 402 and lead 403. The lead 403 includes a paddle body 444 and one or more lead bodies 446. In FIG. 11, the lead 403 is shown having two lead bodies 446. It will be understood that the lead 403 can include any suitable number of lead bodies including, for example, one, two, three, four, five, six, seven, eight or more lead bodies 446. An array of electrodes 433, such as electrode 434, is disposed on the paddle body 444, and one or more terminals (e.g., 560 in FIGS. 13A and 13B) are disposed along each of the one or more lead bodies 446. In at least some embodiments, the lead has more electrodes than terminals.

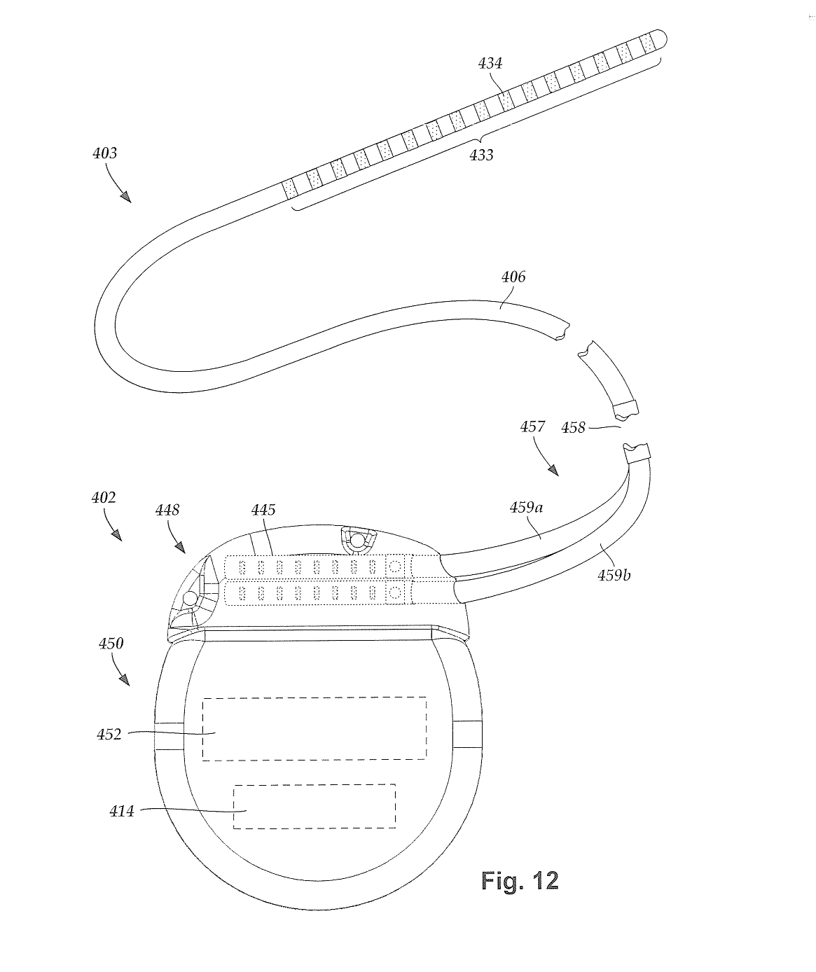

FIG. 12 illustrates schematically another embodiment in which the lead 403 is a percutaneous lead. In FIG. 12, the electrodes 434 are shown disposed along the one or more lead bodies 446. In at least some embodiments, the lead 403 is isodiametric along a longitudinal length of the lead body 446.

The lead 403 can be coupled to the implantable control module 402 in any suitable manner. In FIG. 11, the lead 403 is shown coupling directly to the implantable control module 402. In at least some other embodiments, the lead 403 couples to the implantable control module 402 via one or more intermediate devices (500 in FIGS. 13A and 13B). For example, in at least some embodiments one or more lead extensions 524 (see e.g., FIG. 13B) can be disposed between the lead 403 and the implantable control module 402 to extend the distance between the lead 403 and the implantable control module 402. Other intermediate devices may be used in addition to or in lieu of one or more lead extensions including, for example, a splitter an adaptor, or the like or combinations thereof. It will be understood that, in the case where the electrical stimulation system includes multiple elongated devices disposed between the lead 40 and the implantable control module 402, the intermediate devices may be configured into any suitable arrangement.

In FIG. 12, the electrical stimulation system 400 is shown having a splitter 457 configured and arranged for facilitating coupling of the lead 403 to the implantable control module 402. The splitter 457 includes a splitter connector 458 configured to couple to a proximal end of the lead 403, and one or more splitter tails 459a and 459b configured and arranged to couple to the implantable control module 402 (or another splitter, a lead extension, an adaptor, or the like).

The implantable control module 402 includes a connector housing 448 and a sealed electronics housing 450. An electronic subassembly 452 (which includes the processor 110 (see, FIGS. 1-3) and the power source 414 are disposed in the electronics housing 450. A connector 445 is disposed in the connector housing 448. The connector 445 is configured and arranged to make an electrical connection between the lead 403 and the electronic subassembly 452 of the implantable control module 402.

The electrical stimulation system or components of the electrical stimulation system, including the paddle body 444, the one or more of the lead bodies 446, and the implantable control module 402, are typically implanted into the body of a patient. The electrical stimulation system can be used for a variety of applications including, but not limited to deep brain stimulation, neural stimulation, spinal cord stimulation, muscle stimulation, and the like.

The electrodes 434 can be formed using any conductive, biocompatible material. Examples of suitable materials include metals, alloys, conductive polymers, conductive carbon, and the like, as well as combinations thereof. In at least some embodiments, one or more of the electrodes 434 are formed from one or more of: platinum, platinum iridium, palladium, palladium rhodium, or titanium.

Any suitable number of electrodes 434 can be disposed on the lead including, for example, four, five, six, seven, eight, nine, ten, eleven, twelve, fourteen, sixteen, twenty-four, thirty-two, or more electrodes 434. In the case of paddle leads, the electrodes 434 can be disposed on the paddle body 444 in any suitable arrangement. In FIG. 11, the electrodes 434 are arranged into two columns, where each column has eight electrodes 434.

The electrodes of the paddle body 444 (or one or more lead bodies 446) are typically disposed in, or separated by, a non-conductive, biocompatible material such as, for example, silicone, polyurethane, polyetheretherketone (“PEEK”), epoxy, and the like or combinations thereof. The one or more lead bodies 446 and, if applicable, the paddle body 444 may be formed in the desired shape by any process including, for example, molding (including injection molding), casting, and the like. The non-conductive material typically extends from the distal ends of the one or more lead bodies 446 to the proximal end of each of the one or more lead bodies 446.

In the case of paddle leads, the non-conductive material typically extends from the paddle body 444 to the proximal end of each of the one or more lead bodies 446. Additionally, the non-conductive, biocompatible material of the paddle body 444 and the one or more lead bodies 446 may be the same or different. Moreover, the paddle body 444 and the one or more lead bodies 446 may be a unitary structure or can be formed as two separate structures that are permanently or detachably coupled together.

One or more terminals (e.g., 560 in FIGS. 13A-13B) are typically disposed along the proximal end of the one or more lead bodies 446 of the electrical stimulation system 400 (as well as any splitters, lead extensions, adaptors, or the like) for electrical connection to corresponding connector contacts (e.g., 564 in FIGS. 13A-13B). The connector contacts are disposed in connectors (e.g., 445 in FIGS. 11-13B; and 572 FIG. 13B) which, in turn, are disposed on, for example, the implantable control module 402 (or a lead extension, a splitter, an adaptor, or the like). One or more electrically conductive wires, cables, or the like (i.e., “conductors”—not shown) extend from the terminal(s) to the electrode(s). In at least some embodiments, there is at least one (or exactly one) terminal conductor for each terminal which extends to at least one (or exactly one) of the electrodes.

The one or more conductors are embedded in the non-conductive material of the lead body 446 or can be disposed in one or more lumens (not shown) extending along the lead body 446. For example, any of the conductors may extend distally along the lead body 446 from the terminals 560.

FIG. 13A is a schematic side view of one embodiment of a proximal end of one or more elongated devices 500 configured and arranged for coupling to one embodiment of the connector 445. The one or more elongated devices may include, for example, one or more of the lead bodies 446 of FIG. 11, one or more intermediate devices (e.g., a splitter, the lead extension 524 of FIG. 13B, an adaptor, or the like or combinations thereof), or a combination thereof.

The connector 445 defines at least one port into which a proximal ends 446A, 446B of the elongated device 500 can be inserted, as shown by directional arrows 562a, 562b. In FIG. 13A (and in other figures), the connector housing 448 is shown having two ports 554a, 554b. The connector housing 448 can define any suitable number of ports including, for example, one, two, three, four, five, six, seven, eight, or more ports.

The connector 445 also includes one or more connector contacts, such as connector contact 564, disposed within each port 554a, 554b. When the elongated device 500 is inserted into the ports 554a, 554b, the connector contact(s) 564 can be aligned with the terminal(s) 560 disposed along the proximal end(s) of the elongated device(s) 500 to electrically couple the implantable control module 402 to the electrodes (434 of FIG. 11) disposed on the paddle body 445 of the lead 403. Examples of connectors in implantable control modules are found in, for example, U.S. Pat. Nos. 7,244,150 and 8,224,450, which are incorporated by reference.

FIG. 13B is a schematic side view of another embodiment that includes a lead extension 524 that is configured and arranged to couple one or more elongated devices 500 (e.g., one of the lead bodies 446 of FIGS. 11 and 12, the splitter 457 of FIG. 12, an adaptor, another lead extension, or the like or combinations thereof) to the implantable control module 402. In FIG. 13B, the lead extension 524 is shown coupled to a single port 554 defined in the connector 445. Additionally, the lead extension 524 is shown configured and arranged to couple to a single elongated device 500. In alternate embodiments, the lead extension 524 is configured and arranged to couple to multiple ports 554 defined in the connector 445, or to receive multiple elongated devices 500, or both.

A lead extension connector 572 is disposed on the lead extension 524. In FIG. 13B, the lead extension connector 572 is shown disposed at a distal end 576 of the lead extension 524. The lead extension connector 572 includes a connector housing 578. The connector housing 578 defines at least one port 530 into which terminal(s) 560 of the elongated device 500 can be inserted, as shown by directional arrow 538. The connector housing 578 also includes a plurality of connector contacts, such as connector contact 580. When the elongated device 500 is inserted into the port 530, the connector contacts 580 disposed in the connector housing 578 can be aligned with the terminal(s) 560 of the elongated device 500 to electrically couple the lead extension 524 to the electrodes (434 of FIGS. 11 and 12) disposed along the lead (403 in FIGS. 11 and 12).

In at least some embodiments, the proximal end of the lead extension 524 is similarly configured and arranged as a proximal end of the lead 403 (or other elongated device 500). The lead extension 524 may include one or more electrically conductive wires (not shown) that electrically couple the connector contact(s) 580 to a proximal end 548 of the lead extension 524 that is opposite to the distal end 576. The conductive wire(s) disposed in the lead extension 524 can be electrically coupled to one or more terminals (not shown) disposed along the proximal end 548 of the lead extension 524. The proximal end 548 of the lead extension 524 is configured and arranged for insertion into a connector disposed in another lead extension (or another intermediate device). As shown in FIG. 13B, the proximal end 548 of the lead extension 524 is configured and arranged for insertion into the connector 445.

The embodiments of FIGS. 11-13B illustrate a control module 402 with a connector 445 into which a proximal end portion of the lead or lead extension can be removably inserted. It will be recognized, however, that other embodiments of a control module and lead can have the lead or lead extension permanently attached to the control module. Such an arrangement can reduce the size of the control module as the conductors in the lead can be permanently attached to the electronic subassembly. It will also be recognized that, in at least some embodiments, more than one lead can be attached to a control module.

The above specification and examples provide a description of the manufacture and use of the invention. Since many embodiments of the invention can be made without departing from the spirit and scope of the invention, the invention also resides in the claims hereinafter appended.

What is claimed as new and desired to be protected by Letters Patent of the United States is:

1. An electrical stimulation system, comprising:
an implantable control module configured and arranged for implantation in a body of a patient and comprising an antenna and a processor coupled to the antenna, wherein the control module is configured and arranged to provide electrical stimulation signals to an electrical stimulation lead coupled to the implantable control module for stimulation of patient tissue;
an external programming unit configured and arranged to communicate with the processor of the implantable control module using the antenna and to provide or update stimulation parameters for production of the electrical stimulation signals; and
a patient interface unit configured and arranged to communicate with the external programming unit, the control module, or both, wherein the patient interface unit is configured and arranged to permit a patient to alter one or more of the stimulation parameters within pre-set limits.

2. The electrical stimulation system of claim 1, further comprising a database configured and arranged to receive stimulation data from the external programming unit, wherein the stimulation data comprises at least one of the stimulation parameters.

3. The electrical stimulation system of claim 1, wherein the external programming unit, comprises
a user interface configured and arranged to receive input from a user, and
a processor in communication with the user interface and configured and arranged to perform the following actions:
a) requesting feedback from the patient,
b) receiving patient feedback input at the user interface by the user, and
c) in response to the patient feedback, adjusting the stimulation parameters.

4. The electrical stimulation system of claim 1, wherein the patient interface unit, comprises
a user interface configured and arranged to receive input from a user, and
a processor in communication with the user interface and configured and arranged to perform the following actions:
a) requesting feedback from the patient,
b) receiving patient feedback input at the user interface by the patient, and
c) in response to the patient feedback, adjusting the stimulation parameters.

5. The electrical stimulation system of claim 4, wherein receiving patient feedback comprises receiving verbal or visual feedback from the patient, wherein the user interface is configured and arranged to receive and record the verbal or visual feedback.

6. The electrical stimulation system of claim 4, wherein receiving patient feedback comprises presenting an anatomical depiction on the user interface for the patient to provide feedback using the anatomical depiction.

7. The electrical stimulation system of claim 1, wherein the external programming unit, comprises
a user interface configured and arranged to receive input from a user, and
a processor in communication with the user interface and configured and arranged to perform the following actions:
a) directing stimulation of the patient over a range of the stimulation parameters,
b) receiving patient feedback over the range of the stimulation parameters, and
c) presenting an indication of stimulation effects over the range of the stimulation parameters.

8. The electrical stimulation system of claim 1, where the electrical stimulation system is configured and arranged to determine a figure of merit based on feedback from either the patient or a clinician to electrical stimulation provided by the control module.

9. The electrical stimulation system of claim 8, wherein the electrical stimulation system is configured and arranged to determine a figure of merit based on feedback from both the patient and the clinician to electrical stimulation provided by the control module.

10. An electrical stimulation system, comprising:
an implantable control module configured and arranged for implantation in a body of a patient and comprising an antenna and a processor coupled to the antenna, wherein the control module is configured and arranged to provide electrical stimulation signals to an electrical stimulation lead coupled to the implantable control module for stimulation of patient tissue; and
an external programming unit configured and arranged to communicate with the processor of the implantable control module using the antenna and to provide or update stimulation parameters for production of the electrical stimulation signals, wherein the first external programming unit is configured and arranged to communicate with a database remote from the external programming unit and the implantable control module to store the stimulation parameters at the database for use by other users.

11. The electrical stimulation system of claim 10, further comprising the database remote from the external programming unit and the implantable control module and configured and arranged to store the stimulation parameters.

12. The electrical stimulation system of claim 10, wherein the external programming unit, comprises
a user interface configured and arranged to receive input from a user, and
a processor in communication with the user interface and configured and arranged to perform the following actions:
a) requesting authorization from the patient for use of patient data,
b) receiving authorization from the patient, and
c) in response to the patient authorization, sending patient data to the database.

13. The electrical stimulation system of claim 11, wherein the database, comprises
a user interface configured and arranged to receive input from a user, and
a processor in communication with the user interface and configured and arranged to perform the following actions:
a) requesting authorization patient for use of patient data,
b) receiving authorization from the patient,
c) receiving patient data, and
d) in response to the patient authorization, aggregating the patient data with other data at the database.

14. The electrical stimulation system of claim 11, wherein the database, comprises
a user interface configured and arranged to receive input from a user, and
a processor in communication with the user interface and configured and arranged to perform the following, actions:
a) receiving a patient pain condition,
b) correlating the patient pain condition with aggregated data from a plurality of patients and a plurality of pain conditions, and
c) presenting recommendations for one or more stimulation parameters.

15. The electrical stimulation system of claim 14, wherein correlating the patient pain condition with the aggregated data comprises correlating the patient pain condition with a previously prepared correlation construct generated using the aggregated data.

16. The electrical stimulation system of claim 11, wherein the database contains medical image data as part of the aggregated data.

17. A non-transitory computer-readable medium having processor-executable instructions for providing stimulation recommendations based on aggregated data from a plurality of patients and a plurality of pain conditions, the processor-executable instructions when installed onto a device enable the device to perform actions, comprising:
receiving a patient pain condition;
correlating the patient pain condition with the aggregated data; and
presenting recommendations for one or more stimulation parameters.

18. The non-transitory computer-readable medium of claim 17, wherein the processor-executable instructions when installed onto a device enable the device to perform the following additional actions:
requesting authorization patient for use of patient data;
receiving authorization from the patient;
receiving patient data; and
in response to the patient authorization, aggregating the patient data with other data at the database.

19. The non-transitory computer-readable medium of claim 17, wherein the processor-executable instructions when installed onto a device enable the device to perform the following additional action:
generating a target stimulation map from the aggregated data.

20. The non-transitory computer-readable medium of claim 17, wherein the processor-executable instructions when installed onto a device enable the device to perform the following additional action:
receiving at least one medical image of the patient.
